(19) United States
(12) Patent Application Publication
(10) Pub. No.: US 2024/0018463 A1
(43) Pub. Date: Jan. 18, 2024

(54) FIBER REACTOR SYSTEM AND METHOD

(71) Applicant: LE3 Technology LLC, De Soto, KS (US)

(72) Inventor: Scott A. Lucas, De Soto, KS (US)

(21) Appl. No.: 18/223,356

(22) Filed: Jul. 18, 2023

Related U.S. Application Data
(60) Provisional application No. 63/390,178, filed on Jul. 18, 2022.

(51) Int. Cl. C12M 1/00

(52) U.S. Cl. CPC C12M 47/10; C12M 47/16; C12M 47/18

(57) ABSTRACT

A fiber reactor system includes a fiber slurry tank configured for receiving fiber feed containing cellulose and hemicellulose for forming a fiber slurry, which is mixed with evaporator condensate. The fiber slurry is mixed with steam vapors for sterilizing. A fiber reactor system is configured for receiving ruled fiber and mixing with microorganisms. The scrubber water is fed the top of a scrubber to capture ethanol and an ethanol-laden fiber stream is sent to a low-ethanol beer stripping system.

### CROSS-REFERENCE TO RELATED APPLICATION

This application claims priority in U.S. Provisional Patent Application No. 63/390,178, filed Jul. 18, 2023, which is incorporated herein by reference.

### BACKGROUND OF THE INVENTION

### 1. Field of the Invention

The present invention relates generally to processing cellulose and hemicellulose materials, and in particular to a system and method for producing an ethanol-laden fiber stream for a low ethanol beer stripping system.

### 2. Description of the Related Art

Systems and methods have previously been available for processing cellulose and hemicellulose materials. However, none has been available with the advantages and features of the present invention.

### BRIEF SUMMARY OF THE INVENTION

This invention generally provides a system and method processing cellulose and hemicellulose materials, and in particular to a system and method for producing an ethanol-laden fiber stream for a low ethanol beer stripping system.

### BRIEF DESCRIPTION OF THE DRAWINGS

The drawings constitute a part of this specification and include exemplary embodiments of the present invention and illustrate various objects and features thereof.

FIG. 1 is a schematic diagram of a fiber reactor system embodying an aspect of the present invention.

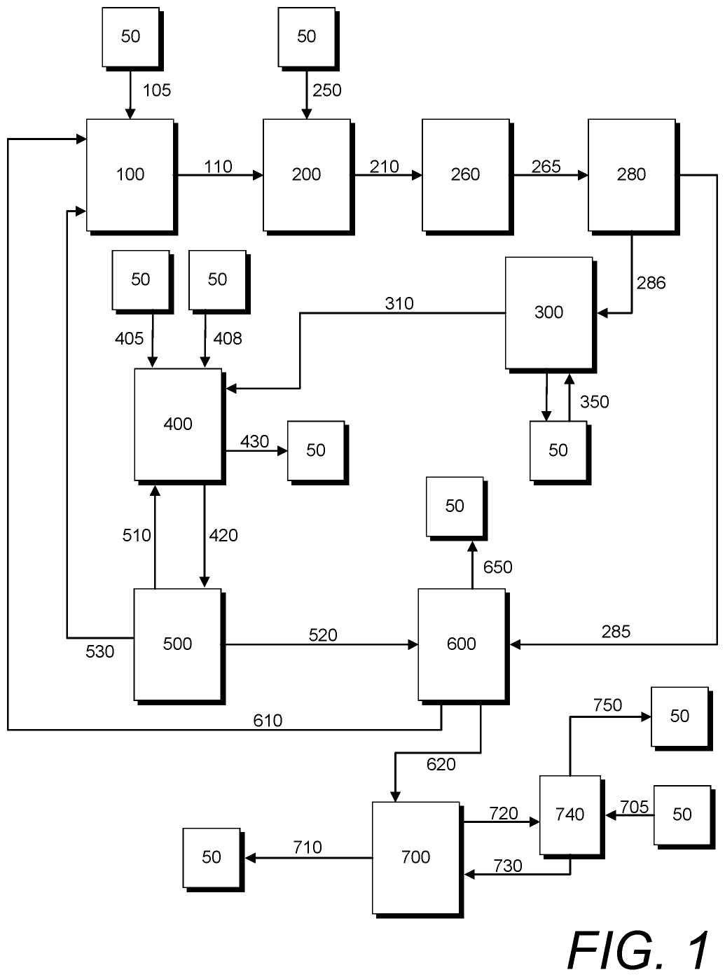

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. Said terminology will include the words specifically mentioned, derivatives thereof and words of similar meaning.

### II. Preferred Embodiment Fiber Reactor System and Method

Fiber feed, 105, containing cellulose and hemicellulose at varying content and milled to less than ¼″ will be fed to the Fiber Slurry Tank, 100, from a preexisting ethanol plant where the material is mixed with evaporator condensate, 610, and recycled fiber, 530, to produce a fiber slurry, 110, that is approximately 5-15% wt solids. This fiber slurry, 110, is fed to the Fiber Sterilization system, 200, where steam 250 is injected in the mixture to produce hot fiber slurry, 210, and fed to a hot holding tank, 260, where the mixture is held at approximately 200 F to 280 F for 5 minutes to sterilize the material. This fiber slurry is mixed with steam vapors where the slurry is heated to 280 F and held for 5 minutes in the Hot Hold Tank, 260, to sterilize the material. The heat-treated fiber slurry, 265, is fed to a Fiber Flash Tank, 280, where flash vapors, 285, at approximately 185 F are vented and utilized at the Evaporation System, 600. The flash cooled fiber slurry, 286, is then passed through a cooling system, 300, where a cooled fiber slurry, 310, is produced at approximately 90 F and sent to the Fiber Reactor System, 400. The fiber slurry is cooled to 90 F using cooling tower water, 350.

At the Fiber Reactor System, 400, the cooled fiber, 310, is mixed with microorganisms, 405, which may consist of Ruminococcus albus, Fibrobacter succinogenes, Bacteroides and Clostridium, rumen culture isolated from sheep and cattle, and micronutrients, 408, which may consist of sources that provide calcium, magnesium, potassium and nitrogen. The stream of reactor slurry, 420, is pumped continuously from the Fiber Reactor System, 400, to a Fiber Filter System, 500, where a suspended solids stream, 510, is separated and sent back to the Fiber Reactor System, 400. A fine particle filtrate, 530, will be removed from the clear liquid, 520, and sent back to the Fiber Slurry Tank, 100. The clear liquid, 520, from the Fiber Filter System, 500, containing approximately 0.5-2% wt sugars is sent to the Evaporation System, 600, where the sugar stream is condensed to produce a sugar product, 620, that is approximately 4-20% wt solids. Flash vapors 285, from the Fiber Flash Tank, 280, at approximately 185 F is used to provide the heat needed at the Evaporation System, 600. Evaporator condensate, 610, is sent back to the Fiber Slurry Tank, 100, where it is mixed with the incoming fiber feed material.

The evaporator vapors will be condensed using cooling tower water, 650. A small fiber waste stream, 430, will be rejected from the Fiber Reactor System.

The sugar product, 620, can be collected, stored and used for other purposes, such as the production of high value protein. In this case, the sugar product will be sent to a fermentation system, 700, where the steam is converted to an ethanol laden fiber stream, 710, that contains between 2% wt and 8% wt ethanol. CO2, 720, will vent from the fermentation system, 700, and will be processed using the CO2 Scrubber System, 740, where scrubber water, 705, is fed to the top of the scrubber to capture ethanol and send this scrubber bottoms, 730, back to the fermenter. A clean CO2, 750, will be vented from the top of the CO2 Scrubber System, 740, and vented to the atmosphere. The ethanol laden fiber stream, 710, is then sent to the Low Ethanol Beer Stripping System.

### III. Conclusion

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. A fiber reactor system, which comprises:
a fiber slurry tank configured for receiving fiber feed containing cellulose and hemicellulose;
said fiber slurry tank being configured for mixing evaporator condensate, recycle fiber to produce a fiber slurry;
said fiber slurry being mixed with steam vapors, heated and held in a hot, whereby said material is sterilized;
said heat-treated fiber slurry being fed to a fiber flash tank configured for flashing papers for utilization in an evaporation system;
the flash-cooled fiber slurry being passed through a cooling system using cooling tower water for producing a cool fiber slurry:
said cool fiber slurry being sent to a fiber reactor system;
a fiber reactor system configured for receiving said cooled fiber, mixing saying with microorganisms, receiving a stream of reactor slurry pump continuously through the fiber reactor system to a fiber filter system; said fiber filter system configured for receiving a suspended solids stream;
said suspended solids streams being separated and sent back to the fiber reactor system;
said fiber filter system including a fine article filter eight;
an evaporation system configured for receiving a sugar stream and condensing same to produce a sugar product;
evaporator condensate being sent back to the fiber slurry tank and mixed with incoming fiber feed material;
evaporator papers being condensed using cooling tower water:
said fiber reactor system rejecting a small fiber waste stream;
a sugar product collected and stored for production of high value, protein in a fermentation system wherein steam is converted to an ethanol-laden fiber stream; and scrubber water being fed to the top of a scrubber to capture ethanol;
scrubber bottoms from said scrubber being sent to a pre-fermenter;
clean CO2 is vented from the top of the CO2 scrubber system to the atmosphere; and
an ethanol-laden fiber stream being sent to a low ethanol beer stripping system.
